(19) United States
(12) Patent Application Publication
(10) Pub. No.: US 2019/0029684 A1
(43) Pub. Date: Jan. 31, 2019

(54) ANASTOMOSIS CONNECTING DEVICES

(71) Applicant: SURG SOLUTIONS, S.L., Barcelona (ES)

(72) Inventors: Jordi DESCARREGA, BARCELONA (ES); Joan FONTDEVILA FONT, BARCELONA (ES); German HEBER BELLEMMI, VILADECANS (ES)

(73) Assignee: SURG SOLUTIONS, S.L., Barcelona (ES)

(21) Appl. No.: 16/071,853

(22) PCT Filed: Nov. 21, 2016
(86) PCT No.: PCT/EP2016/078352

§ 371 Date: Jul. 20, 2018

(30) Foreign Application Priority Data
Jan. 22, 2016 (EP) 16382027.7

(51) Int. Cl. A61B 17/11

(52) U.S. Cl. CPC A61B 17/11; A61B 2017/00526

(57) ABSTRACT

An anastomosis device having a tubular structure with first and second tubular end portions and an intermediate portion, the intermediate portion being united at one end to the first tubular end portion and at the other end to the second tubular end portion, the first and the second tubular end portions having expansion elements to allow the expansion and contraction of each end portion in at least one radial direction thereof, and the intermediate portion having approximation elements configured and attached to the tubular end portions to allow the length in the axial direction of the intermediate portion to increase and decrease.

The present disclosure is related to anastomosis devices.

### BACKGROUND

In surgical procedures it is often necessary to perform vascular anastomosis, for example joining blood vessels end-to-end.

Anastomosis is traditionally done by manual surgical suture. However, manual suture is technically complex, and therefore it has a long learning curve, a significant degree of failures and a reliability that varies from one surgeon to another. Furthermore, it is a slow operation, which adds to ischemia time and to the overall time of the surgical procedure.

A number of devices have been developed for simplifying and increasing the reliability of manual suture, for example, there are known stent-like devices which are introduced into the vessels in a radially compressed condition, and then expanded so they contact the vascular endothelium and maintain the vessel open. A clip is then fitted to press the vessels against the expanded stent, to seal the interface and avoid leaks.

Other known devices have included two interlocking sleeves, each of them provided with projections to engage one of the vessels. Each sleeve is attached to one of the vessels to be joined, and the two sleeves are then interlocked to seal the joint.

A system and method for using a tissue scaffold to facilitate healing of an anastomosis has been disclosed to provide a tissue scaffold for placement at an anastomotic site within a body lumen having a radially expandable scaffold structure having lateral and mid portions, at least one retention element coupled to each lateral portion and a barrier layer.

A medical graft component and methods of installing same have been disclosed for securing an axial end portion of a tubular graft conduit in a lumen of a patient's existing tubular body organ structure via an aperture in a side wall thereof. An anchor device is configured for attachment to the end portion of the tubular graft conduit. The anchor device defines a constant axial length and a cross-section radially expandable.

An anastomosis member has been disclosed having a generally cylindrical body with a plate member to be brought into contact with first and second blood vessels. The plate member is arranged in contact with the first and the second blood vessels at an anastomosed site where the first and the second blood vessels are anastomosed to each other. The plate member is provided with a plurality of protrusions which are engaged with at least one of the first and the second blood vessels to avoid the dislocation of the first and the second blood vessels at the anastomosed site.

However, the known devices do not provide satisfactory solutions in all cases, and especially in the case of an anastomosis performed in microsurgery procedures, such as microvascular free flap surgery, which is where manual suture is more complex.

### SUMMARY

The present anastomosis devices and methods may be used in microsurgical procedures and also, in general, surgery of vascular anastomosis.

An anastomosis device according to the present disclosure may have a tubular structure, said structure including first and second tubular end portions and an intermediate portion, the intermediate portion being united at one end to the first tubular end portion and at the other end to the second tubular end portion, the first and the second tubular end portions having expansion elements to allow the expansion and contraction of each end portion in at least one radial direction thereof, and the intermediate portion having approximation elements configured and attached to the tubular end portions in such a way that they allow the length of the intermediate portion to increase and decrease in the axial direction.

The length in the axial direction of the intermediate portion may increase and decrease and the first and second end portions may compress and expand. This configuration allows inserting the device in compressed condition into the ends of two vessels that are to be anastomosed, and then causing the end portions to expand, and become attached to the ends of the two vessels, and in the same operation causing the intermediate portion to decrease in length such as to pull the ends of the vessels towards each other.

The anastomosis procedure is therefore simplified, because it requires less skill and less time, with all the advantages that this brings about. In certain circumstances it may allow completely avoiding the need for manual suture, because the blood vessels are held together by the device and become joined without the need of suture.

Furthermore, the configuration of the device is such that it can have a small size, suitable in microsurgery applications.

In implementations of such a device the expansion elements may be arranged to allow the expansion and contraction of each end portion in a first radial direction while maintaining substantially constant the dimension of each end portion in a second radial direction perpendicular to the first direction. This may make easier the initial attachment of the tubular end portions to the inner surface of the lumen.

In some implementations, at least the expansion elements of the first and second tubular end portions may be made of a shape-memory material. This material may be such that the expansion elements can be deformed to compress the end portions in said at least one radial direction at a temperature lower than the corporal temperature, and recover from the deformation to expand again the end portions upon heating. Use of a shape-memory material thus allows the tubular end portions of the device to expand after insertion of the device, without the need of applying an external force.

In some implementations, the approximation elements of the intermediate portion may have at least one X-shaped unit. Such a unit may be attached to the tubular end portions in such a way that it increases or decreases the axial length when the tubular end portions expand or contract, respectively. An approximation element having a wave shaped assembly, may be foreseen, for a greater change in axial dimension.

In some implementations, the tubular end portions also have at least two plates, configured to contact the inner surface of a lumen, with the longitudinal edges of two consecutive plates being connected to each other through the expansion elements. Thus, in the tubular end portions the expansion elements have the function of allowing the expansion and contraction, while the plates have the function of contacting and engaging the endothelial tissue of the lumen, such as to allow dragging the lumen axially along with the tubular end portions, without causing any damage to the tissue.

In some implementations, each of the first and the second tubular end portions may be configured to be rolled up on themselves. This may obtain better seal of end portions when they expand in a radial direction.

In another aspect, a method of manufacturing an anastomosis device is provided. The method of manufacturing may include:
providing a tubular structure;
cutting the tubular structure to form first and second tubular end portions and an intermediate portion having approximation elements which may be configured, and attached to the tubular end portions, in such a way that they allow the length of the intermediate portion to increase and decrease in the axial direction;
activating the tubular structure to cause a contraction of said first and second tubular end portions in at least one radial direction and to increase the length of said intermediate portion in an axial direction.

Thus, an anastomosis device as herein described may be obtained in an easy manner.

### BRIEF DESCRIPTION OF THE DRAWINGS

Some non-limiting examples of the present disclosure will be described in the following with reference to the appended drawings, in which:

FIGS. 1a, 1b, 1c are views in perspective, side view and cross section of a first implementation of an anastomosis device as disclosed herein, in an activated position;

FIGS. 2a, 2b, 2c and are views in perspective, side view and cross section of the first implementation of the device, in an inactivated position;

FIGS. 3a, 3b, 3c are schematic diagrams illustrating the use of a device as disclosed herein, for example that shown in FIGS. 1a-1c, 2a-2c, in a vascular end-to-end anastomosis;

FIGS. 4a, 4b and 5a, 5b are views in perspective of a second implementation of an anastomosis device as disclosed herein, in activated and inactivated position, respectively;

FIG. 6a is a schematic view of an alternative implementation of the approximation elements of the intermediate portion of a device as disclosed herein;

FIGS. 6b and 6c show the use of a device such as that of FIG. 6a in a vascular end-to-end anastomosis;

FIG. 7 is a diagram showing the steps of a method for end-to-end vascular anastomosis according to implementations disclosed herein; and

FIGS. 8a, 8b are schematics views in perspective of a further alternative implementation of an anastomosis device as disclosed herein, in activated and inactivated position, respectively;

In order to show and facilitate understanding of the features of the devices disclosed herein, at least some of the attached figures may not be to scale.

### DETAILED DESCRIPTION OF EXAMPLES

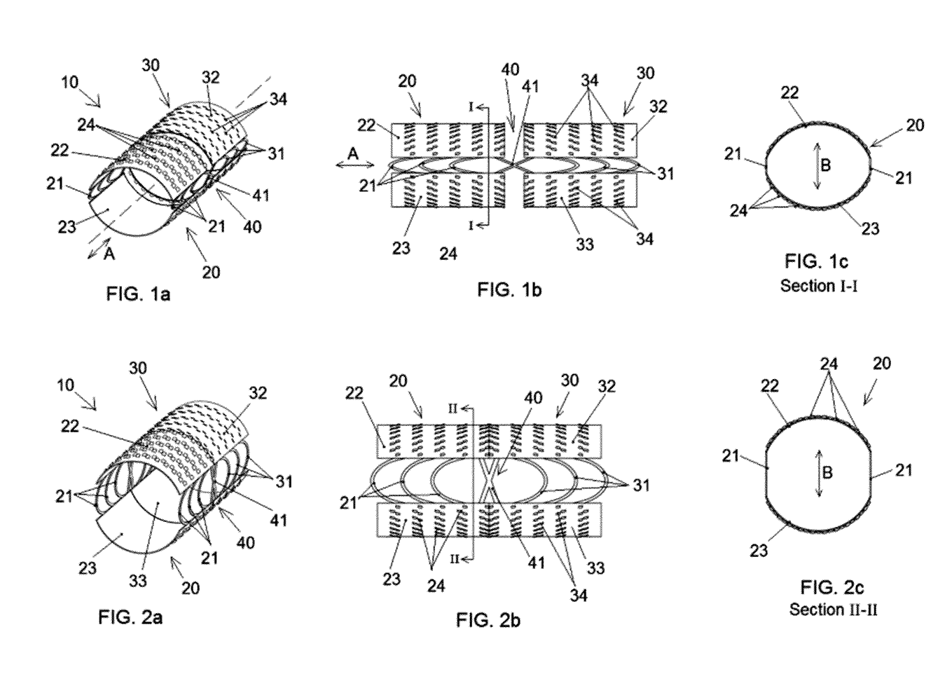

Implementations of anastomosis devices disclosed herein may have, as shown in the example of FIGS. 1a-1c, a tubular structure 10 with a first tubular end portion 20 and a second tubular end portion 30, and an intermediate portion 40, united to the first and second tubular end portions 20, 30. As may be seen in the Figs., the tubular end sections 20 and 30 may be coaxial.

Each of the tubular end portions 20, 30 may have expansion elements 21, 31, which allow the expansion and contraction of the end portions in at least one radial direction of the tubular structure, as will be described later on.

In the implementation shown in FIGS. 1a-1c, 2a-2c, the expansion elements 21, 31 are struts that adopt a C-shape, with the ends of the C-shape more or less close to each other depending on the state of compression or expansion, as shown in FIGS. 1a, 1b and 2a, 2b. However, the expansion elements 21, 31 can be any other shape suitable for the desired expansion and contraction of the two tubular end portions 20, 30.

Each of the first and second tubular end portions 20, 30 may have at least two curved plates 22, 23 and 32, 33 respectively, which are arranged to form part of the surface of the tubular end portions 20, 30 and are configured to contact the inner surface of a lumen, for example a lumen of a blood vessel, as will be described later on, upon expansion of the tubular end portions 20, 30. The plates 22, 23 and 32, 33 may be substantially stiff.

The longitudinal edges of consecutive or adjacent plates 22, 23 are connected to each other through the expansion elements 21, 31, as visible in the Figs., such that they complete the tubular structure of the first end portion 20. The same occurs with the second end portion 30.

In this implementation of the device, the expansion and contraction of the two tubular end portions 20, 30 occurs only in one radial direction B, as visible in FIGS. 1c and 2c, while the dimension, width or diameter of each tubular end portion 20, 30 in the radial direction that is perpendicular to direction B remains substantially constant upon expansion or contraction.

The intermediate portion 40 may have approximation elements 41, attached to the tubular end portions 20, 30. In the embodiment of FIGS. 1a-1c, 2a-2c, the approximation elements 41 are X-shaped. For example, as shown in the Figs., each approximation element 41 is formed by an X-shaped strut having two legs attached to the first tubular end portion 20 and the other two legs attached to the second tubular end portion 30.

More generally, the approximation elements 41 may have another configuration, and may be attached to the tubular end portions in other ways, provided that the configuration and attachment allow that the length of the intermediate portion, in the axial direction A of the tubular structure, increases and decreases.

As a result, the approximation elements 41 may keep the two tubular end portions 20, 30 apart when the device is being inserted in the intended position and the two tubular end portions 20, 30 are in a radially contracted position (FIGS. 1a, 1b, 1c), and they may pull the two tubular end portions 20, 30 together after the device is inserted, substantially in the same operation in which the tubular end portions 20, 30 are expanded in a radial direction (FIGS. 2a, 2b, 2c).

In the present specification, expansion elements and approximation elements may be elements that do not break, or elements that do not undergo a permanent, non-recoverable deformation when their shape is changed to the degree needed for their operation as disclosed above. They may be for example elements showing an elastic or super-elastic behaviour, and/or of shape-memory materials.

FIGS. 1a-1c and 2a-2c also show that the first and second tubular end portions 20, 30 may have projections 24, 34 on at least part of their outer surface, for example on the plates 22, 23 and 32, 33. Projections 24, 34 (spikes, surface roughness, or other) are configured to contact and engage the inner surface of a lumen, for example the endothelium of a blood vessel, such as to be able to drag the vessel along in the movements of one of the tubular end portions 20, 30.

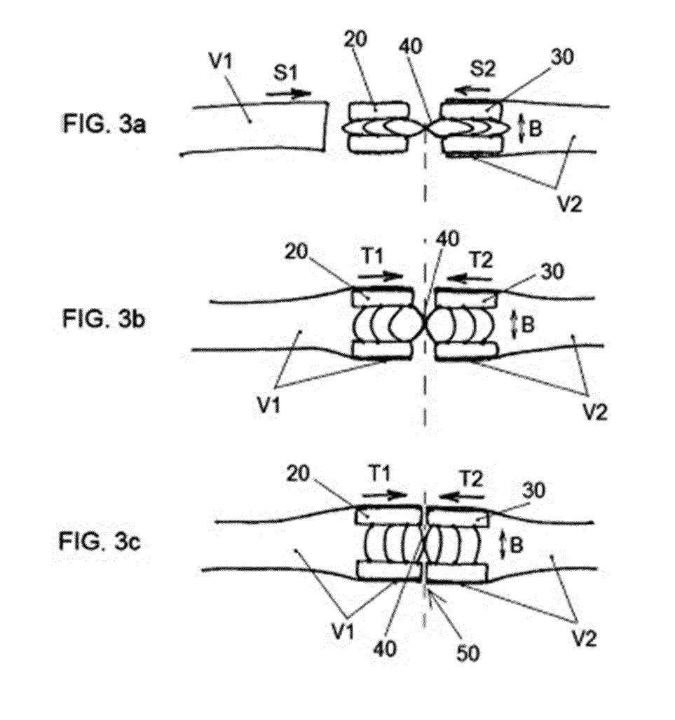

The use of an anastomosis device such as disclosed above will now be described with reference to FIGS. 3a, 3b, 3c, which show three steps of an end-to-end vascular anastomosis procedure according to an implementation disclosed herein.

Initially, in FIG. 3a, the device is in a position described herein as an “activated” position, i.e. a position ready to use wherein the expansion elements 21, 31 are compressed, the two tubular end portions 20, 30 are contracted in radial direction B, and the intermediate portion 40 maintains the two portions 20, 30 at a distance from each other.

With the device in this activated condition, two blood vessels V1 and V2 are brought sliding on the tubular end portions 20, 30 in the direction of the arrows S1 and S2. The size of the device is selected such that in the radial direction B the lumen of the vessels is slightly larger than the dimension of the tubular end portions 20, 30 in contracted condition, while in the radial direction perpendicular to B the dimensions of the lumen and the tubular end portions 20, 30 are similar.

Once the two vessels V1 and V2 are placed around the tubular end portions 20, 30 respectively, these two portions 20, 30 may start to expand in the radial direction B as a consequence of the expansion elements and they may start getting closer in the direction A by the action of the approximation elements.

By virtue of the dimensions of the tubular end portions 20, 30 and the lumens of the vessels V1, V2, as soon as the vessels are inserted and the device starts to expand, the longitudinal edge region of the plates 22, 23, 32, 33 of the tubular end portions 20, 30 that is closer to the expansion elements 21, 31 contacts and engages the endothelial wall of the lumens through the projections 24, 34, such that the vessels start being dragged along in the movement of the tubular end sections 20, 30, as they expand in the direction B and move towards each other in the direction A.

FIG. 3b shows a step of the procedure where the end portions 20, 30 are in a partly expanded condition. At this point, by virtue of this expansion, most of the surface of the plates 22, 23 and 32, 33 is engaging the lumens of the vessels V1 and V2.

In substantially the same operation, or substantially simultaneously, the length in the axial direction A of the intermediate portion 40 has decreased and therefore reduced the distance between the two end portions 20, 30 and has brought the two vessels V1 and V2 towards each other (arrows T1, T2).

FIG. 3c illustrates a position near the end of the movement of the two end portions 20, 30 towards each other induced by the intermediate portion 40. In this position, the ends of the vessels V1 and V2 are almost in contact with each other, and have been expanded by the expansion of the end portions 20, 30.

At the end of the movement, the vessels V1, V2 may be brought in complete contact with each other at an anastomosis site 50, and the device is in a position described herein as an “inactivated” position, i.e. a position wherein the expansion elements 21, 31 are expanded, the two tubular end portions 20, 30 are expanded in radial direction B, and the intermediate portion 40 maintains the two portions 20, 30 close to each other.

The two vessels are thereafter maintained in position by the device, and this results in anastomosis of the vessels. This may occur without the need of any suture at all, or with a much smaller amount of suture with respect to manual suture anastomosis methods.

Thus, it will be appreciated from the above description that implementations of a device as disclosed herein allow multiple advantages such as fast anastomosis procedures, which do not require a high degree of skill from the surgeon, and also higher safety and reliability in the procedure, with a reduction of the risks of leaks, thrombosis, etc.

Furthermore, the device may be small in size and therefore suitable also for microsurgery applications, wherein manual suture is particularly complex. It may also be simple in construction and therefore relatively low-cost with respect to known devices such as couplers.

According to some implementations, the expansion elements of the first and second tubular end portions of devices disclosed herein are made of a shape-memory material. In some implementations, the approximation elements of the intermediate portion may be made of a shape-memory material.

The shape-memory material is such that the expansion elements can be deformed to compress the end portions in said at least one radial direction at room temperature, and recover from the deformation to expand again the end portions in said at least one radial direction, e.g. upon heating.

Similarly, approximation elements made of shape-memory material can be stretched at room temperature, and recover from this deformation to the initial shape, e.g. upon heating, thus shortening again the intermediate portion.

In some cases, especially in devices of particularly small size such as those intended for microsurgery, the heat of the body may be sufficient for the expansion elements and approximation elements to recover their initial shape.

In other cases, it is foreseen to apply a source of external heating such as a heating fluid, in order to induce the shape recovery.

The shape-memory material may be a suitable alloy or polymer, for example a Nitinol alloy, or poly-L-lactide (PLLA).

In other implementations of the device, the expansion elements of the first and second tubular end portions, and/or the approximation elements of the intermediate portion, may be made of an elastic material such as spring steel.

According to some implementations, the anastomosis device may be made of absorbable materials.

It may be foreseen to provide an anastomosis device such as disclosed that is also drug-eluting, e.g. coated with a suitable substance such that after the procedure it may deliver the substance at and around the anastomosis site.

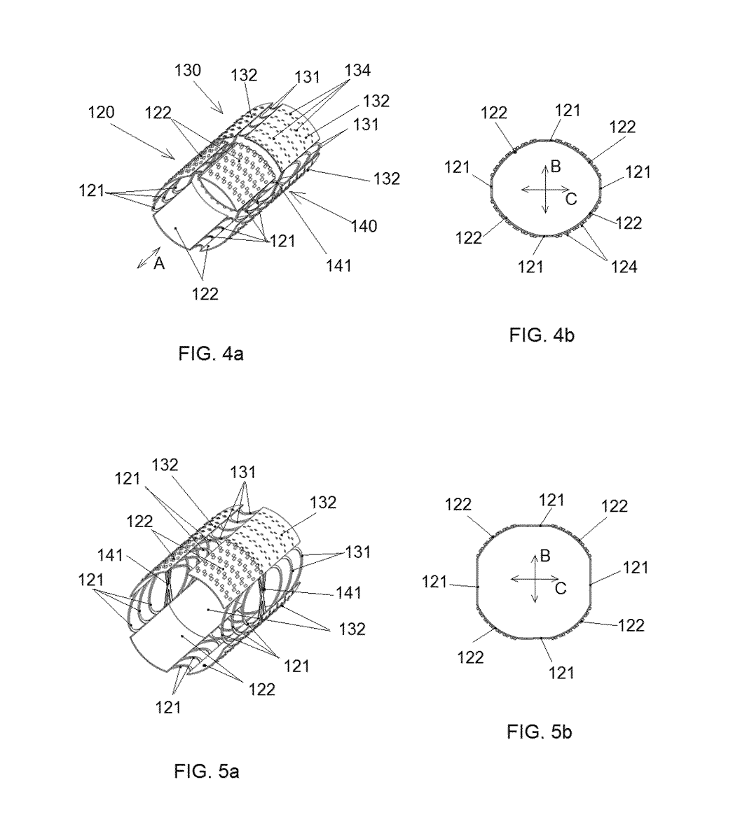

FIGS. 4a, 4b and 5a, 5b illustrate another implementation of an anastomosis device as proposed herein, where FIGS. 4b and 5b are cross sections through one of the tubular end portions of the device that is shown in perspective in FIGS. 4a (in activated position) and 5a (inactivated position).

In this case, each tubular end portion 120, 130 has four plates 122, 132, and four sets of expansion elements 121, 131 between them, as shown in the Figs.

The expansion elements 121 of the first tubular end portion 120 may be attached to the plates 122 such as to bend in an opposite direction with respect to that of the expansion elements 131 of the second tubular element 130 attached to the plates 132; that is, expansion elements 121 and 131 may be oriented in opposite directions.

Furthermore, in each of the tubular end portions 120 and 130 the set of expansion elements between two plates may be oriented in one direction, and the set of expansion elements between the next two plates may be oriented in the opposite direction, as visible in FIGS. 4a and 5a.

It will be appreciated that with the configuration of four plates and four sets of expansion elements, the tubular end portions 120 and 130 will expand and contract in two perpendicular radial directions B and C (see FIGS. 4b and 5b), and not in only one direction as the implementation of FIGS. 1a-1c, 2a-2c.

However, depending on the geometry and/or properties of the expansion elements 121, 131 in each set, it is possible to cause a larger expansion in one direction than in the other.

This implementation also includes approximation elements 141 in an intermediate portion 140, which like in other implementations are configured and attached to the tubular end portions 120, 130 in such a way that the length of the intermediate portion 140 increases or decreases when stretching out the device.

In some implementations the approximation elements 141 may be present between each two consecutive or adjacent plates 122 or 132 (four approximation elements 141), but they may also be present only between two sets of plates 122 or 132, as shown in FIGS. 4a and 5a.

Like in other embodiments, the surface of the first and second tubular end portions may have projections 134 on the outer surface of the plates 122, 132, to assist in the engagement of the tubular end portions with the endothelial tissue of a lumen. In any of the implementations, the projections on the tubular end portions may be spike-shaped and inclined with respect to the axial direction of the tubular structure, although they may also be perpendicular.

In some implementations the projections 34, 134 on each of the first and second tubular end portions 20, 120, and 30, 130 are inclined in opposite directions with respect to the axial direction of the tubular structure, such that, they are suitable to promote insertion of the tubular end portions into the ends of a blood vessel or other lumen, and to oppose its withdrawal.

In this manner, the spikes do not hinder the insertion of the tubular end portions into the ends of two blood vessels or other lumens, and after insertion they provide very good attachment to pull the two vessels towards each other.

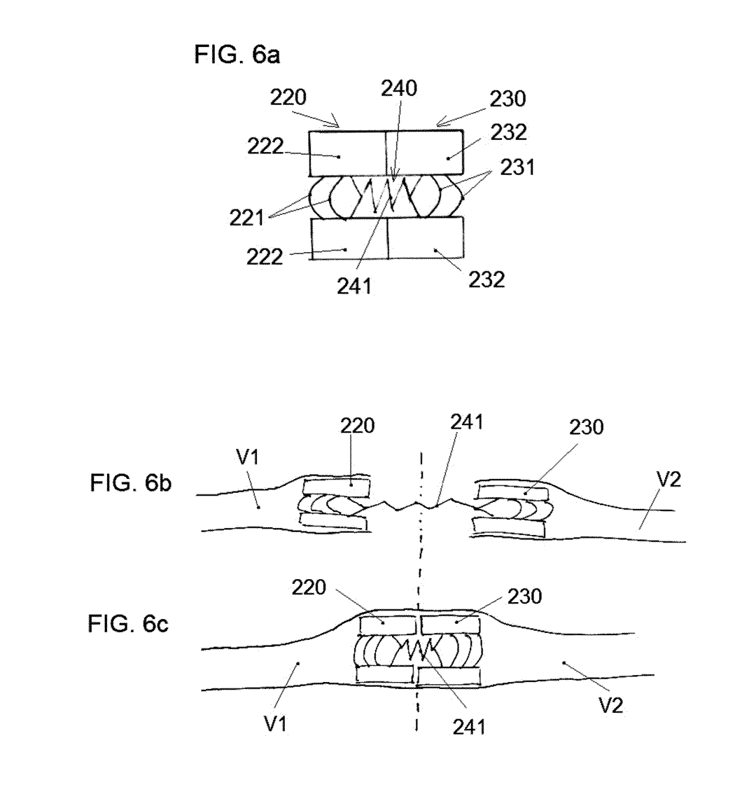

FIG. 6a shows very schematically a further implementation of an anastomosis device, in inactivated condition, with tubular end portions 220, 230 fully expanded and substantially in contact with each other, and an intermediate portion 240. As in previous implementations, each tubular end portion 220, 230 has plates 222, 232 and expansion elements 221, 231, respectively, and the intermediate portion 240 has an approximation element 241, in this case a wave shape. The device may for example have two or four plates in each end portion, and configurations of the expansion elements and the approximation elements and of the projections for attachment to the endothelial tissue as disclosed above for previous examples of the device.

In this implementation the approximation elements 241 of the intermediate portion 240 have a different configuration, allowing an amplification of the axial travel of the two tubular end portions, i.e. a larger increase and decrease of the axial length of the device, and therefore a larger approximation of the blood vessels to be anastomosed, with the same radial expansion of the tubular end portions. This amplification may be appreciated in the views of FIGS. 6b and 6c, which show a device such as that of FIG. 6a in two positions of a vascular end-to-end anastomosis procedure: a position in which the two tubular end portions 220 and 230 have been inserted in two vessels V1 and V2 but have not yet expanded (FIG. 6b), and a position after expansion (FIG. 6c).

The approximation elements of the intermediate portion may be wave shaped, as shown in the Figs.

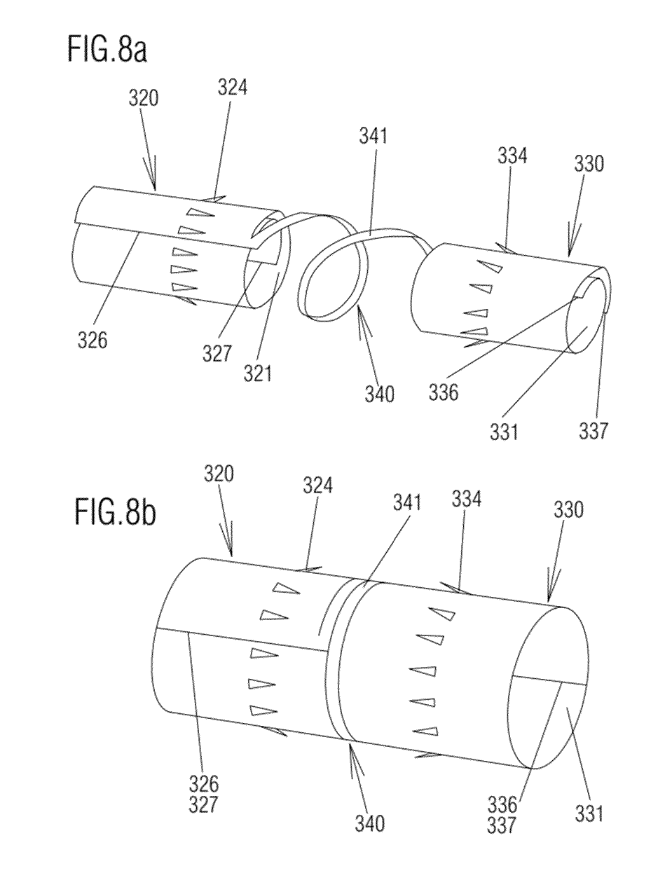

FIGS. 8a, 8b are views in perspective of a further alternative implementation of an anastomosis device as disclosed herein, in activated and inactivated position, respectively. In the examples illustrated in FIGS. 8a, 8b each of the first and the second tubular end portions 320, 330 may be configured to be rolled up on themselves, particularly about the longitudinal axis of the device, such that the first and second tubular end portions 320, 330 may be compressed in at least one radial direction. The tubular end portions 320, 330 may be rolled up in this way in an activated position shown in FIG. 8a. When the device is employed to perform an anastomosis, the first and the second tubular end portions 320, 330 may roll out, particularly about the longitudinal axis, such that the first and second tubular end portions 320, 330 may expand in at least one radial direction (FIG. 8b).

To achieve the aforementioned rolling up and rolling out of the first and second tubular end portions 320, 330 those end portions may have a region 321, 331 made of shape-memory material. The shape-memory material may be any of the herein disclosed ones.

The first and second tubular end portions 320, 330 may have a region 321, 331 that encompasses the full extent of the end portions or only a part thereof. Other implementations of expansion elements herein disclosed may be envisaged in combination with end portions 320, 330.

According to some implementations, the first and second tubular end portions 320, 330 may be formed from a sheet (i.e. laminar configuration element) of material which may be rolled up and out. A longitudinal or axial edge 326, 336 of the sheet may be rolled up inside another longitudinal or axial edge 327, 337 of the sheet as per FIG. 8a. Details of a manufacturing method will be provided later on.

In FIG. 8b both pairs of edges 326, 327 and 336, 337 are not respectively aligned with each other in the inactivated position.

End portions with good sealing may be obtained through the example of FIGS. 8a, 8b owing to the pairs of edges 326, 327 and 336, 337 (at least one pair) which may be substantially abutted to or against each other in the inactivated position.

In FIGS. 8a, 8b projections 324 and 334 are shown which may have the same features as any of the aforementioned implementations, to assist in the engagement of the tubular end portions with the endothelial tissue of a lumen. Then those projections 324, 334 provide end portions of the implementations of FIGS. 8a, 8b with a good attachment to pull two vessels towards each other.

According to another implementation, the approximation element 341 of the intermediate portion 340 may have a helical-shaped unit. In the activated position as per FIG. 8a the approximation element 341 shows a longitudinally extended configuration where the length of the intermediate portion 340 may reach its maximum. In the inactivated position shown in FIG. 8b, the length of the intermediate portion 340 may decrease such that the end portions are pulled towards each other. The helical-shaped unit may be made of shape-memory material as disclosed herein.

Other implementations of the approximation element may be envisaged in combination with the end portions 320, 330.

The implementation illustrated in FIGS. 8a, 8b may allow a clearance at least between the longitudinal edges 326, 327 and 336, 337 so narrow that the risk of leakage may be even reduced in an inactivated condition. The same may apply to the clearance between each of the tubular end portions 320, 330 and the intermediate portion 340 and between each pair of turns of the helical structure of the intermediate portion 340. A better sealing may be achieved in an inactivated condition of the tubular end portions 320, 330 and the intermediate portion 340.

According to an implementation not shown at least one of the pairs of longitudinal edges 326, 327 and 336, 337, edges between each tubular end portion 320, 330 and the intermediate portion 340, and between each pair of turns of the helical structure of the intermediate portion 340, may have a tongue-and-groove joint or a bevel joint for providing an even better sealing.

According to another implementation not shown the device may be coated for instance with a biocompatible polymeric material so as to obtain an even more fluid-tight sealing in any of the above mentioned clearances.

According to a further implementation not shown a ring-like member may be provided around each vessel at the anastomosis site and may be located in such a way that a vessel may be outwardly surrounded by a ring-like member and inwardly by the anastomosis device. The ring-like member may have at least two separable halves with a C-shaped cross section in order to facilitate the installation. Sealing of the anastomosis site may be even enhanced by the use of the ring-like members pressing the vessel onto the anastomosis device.

Implementations of devices as disclosed above may be applied in any kind of anastomosis, and in particular any kind of vascular anastomosis; the tubular end portions may therefore have a diameter, when fully expanded, of between 0.7 mm and 40 mm.

However, the field wherein these devices may afford more advantages is that of microsurgical vascular anastomosis, and in this case the tubular end portions may therefore have a diameter, when fully expanded, of between 0.7 mm and 4 mm.

In the inactivated condition of the device, i.e. the condition wherein the expansion of the tubular end portions is maximum, each region between two consecutive plates of the tubular end portions, has a width of between 10% and 60% of the maximum diameter of the tubular end portions.

For example, in implementations with two plates and two sets of expansion elements, such as that of FIGS. 1a-1c and 2a-2c, each region between two consecutive plates may have a width, when the expansion elements are fully expanded, of about 40% of the maximum diameter of the tubular end portions.

In implementations with four plates and four sets of expansion elements, such as that shown in FIGS. 4a-4b and 5a-5b, each region between two consecutive plates has a width, when the expansion elements are fully expanded, of about 30% of the maximum diameter of the tubular end portions.

In some examples the diameter of one of the tubular end portions may be larger than the diameter of the other.

In the activated condition of the device, i.e. the condition wherein the expansion of the tubular end portions and of its expansion elements is minimum and they are spaced apart from each other, the intermediate portion may have a length, in the axial direction of the device, of between 15% and 50% of the maximum diameter of the tubular end portions.

The length of each of the tubular end portions, in the axial direction of the device, may be of between 2 and 7 times the expanded diameter of the tubular end portions (in this regard, the drawings in the attached Figs. are not to scale: the tubular end portions may generally be longer than shown).

Each tubular end portion 20, 30, 120 or 130 described above may have for example between one and eight expansion elements 21, 31, 121, 131, for example between two and four expansion elements, between each two consecutive or adjacent plates, depending on the length and requirements of each particular device.

The number of expansion elements 21, 31, 121, 131 may not be the same between all the pairs of plates: for example, in the implementation shown in FIGS. 4 and 5 there are three expansion elements 122, 132 between plates 122, 132 where also an approximation element 141 is present, while there are four expansion elements 121, 131 between plates where there is no approximation element 141.

Devices such as disclosed herein may be manufactured in a single part, for example by laser cutting from a tube of a Nitinol alloy or other suitable material, or may be assembled from different parts of the same or of different materials, for example by welding.

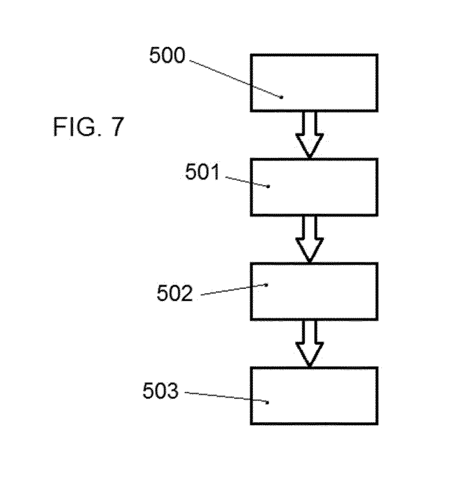

The present disclosure also relates to a method for end-to-end vascular anastomosis. Such a method is illustrated in FIG. 7, and may include:
In block 500, providing an anastomosis device as disclosed in any of the above implementations;
In block 501, activating the device, i.e. causing the first and second tubular end portions to be compressed in the at least one radial direction and the length of the intermediate portion to increase in axial direction;
In block 502, inserting the first and second tubular end portions respectively in a first and second blood vessels to be anastomosed to each other; and
In block 503, inactivating the device, i.e. allowing the first and a second tubular end portions to expand in said at least one radial direction and allowing the length of the intermediate portion to decrease such that the end portions are pulled towards each other.

After a first degree of expansion of the first and a second tubular end portions, at least part of the outer surface of these tubular end portions engages and becomes attached to the endothelial tissue of the first and second vessels (as described e.g. in relation to FIG. 3b above), and during subsequent expansion of the tubular end portions and the decrease in length of the intermediate portion, the first and second vessels are pulled towards each other (FIG. 3c).

Several options are foreseen for the activation of the device, i.e. for applying a force to compress the tubular end portions and increase the length of the intermediate portion, and also for the inactivation of the device, i.e. for allowing or causing the expansion of the tubular end portions and the axial shortening of the intermediate portion.

For example, the device may be activated immediately before it has to be employed in a vascular anastomosis procedure.

Alternatively the activation may be performed as a step at the end of the manufacturing process, and the device may then be packaged and stored in activated position until it is used.

If it is activated immediately before use, the activation may be performed manually by the surgeon or other medical practitioner, or by using a suitable tool.

If the material of at least part of the expansion elements and/or approximation elements is a shape memory material, the activation of the device is performed at a suitable temperature, lower than the body temperature, for example lower than about 25° C.

If the activation is done upon manufacture, the device is thereafter handled and stored in suitable conditions, e.g. suitable temperature conditions, to avoid its inactivation before use.

The inactivation of the device after the two tubular end portions have been inserted in the corresponding lumens may occur when the body temperature heats the device and the expansion elements and/or approximation elements of shape memory material recover their initial shape. However, it is also possible to increase or decrease the speed of inactivation by contacting the device with hot or cold serum, or a similar heat or cold source.

The present disclosure also relates to a method of manufacturing an anastomosis device. That method may include:
providing a tubular structure, such as a tube or the like;
cutting the tubular structure to form first and second tubular end portions 20, 120, 220, 320, 30, 130, 230, 330 and an intermediate portion 40, 140, 240, 340 having approximation elements 41, 141, 241, 341 configured, and which may be attached to the tubular end portions, in such a way that they allow the length of the intermediate portion to increase and decrease in the axial direction;
activating the tubular structure to cause a contraction of said first and second tubular end portions in at least one radial direction and to increase the length of said intermediate portion in an axial direction.

In use in performing an anastomosis, the tubular structure may be allowed to inactivate, to cause an expansion of said first and second tubular end portions in at least one radial direction and to decrease the length of said intermediate portion in an axial direction. The expansion of the end portions and decreasing of the length of the intermediate portion may be achieved substantially simultaneously or separately.

In some implementations like those illustrated in FIGS. 8a and 8b, cutting the tubular structure may include:
a linear cut along the surface of the tubular structure in the axial direction which may extend from a first end to a first point of the tubular structure. By this operation the edges 326 and 327 of the first tubular end portion 320 may be obtained;
a linear cut along the surface of the tubular structure in the axial direction which may extend from a second end to a second point of the tubular structure, the second end being opposite to the first end (in the axial direction for instance). By this operation the edges 336 and 337 of the second tubular end portion 330 may be obtained;
a helical-path cut around the longitudinal axis, such that the helical-path cut may be provided between the first and second end of the tubular structure. By this operation a helical-shaped unit of the approximation element 341 of the intermediate portion 340 may be obtained, the approximation element 341 being expandable in axis direction;

The end portions may be joined to each other through the intermediate portion.

The number of cuts may vary and also the number of performed operations depending on the configuration of the anastomosis device. The cuts may be carried out using a laser beam.

In some implementations like those illustrated in FIG. 8a, activating the tubular structure may further include:
rolling the first and second tubular end portions up on themselves, so as to contract the end portions in at least one radial direction thereof;
pulling the first and second tubular end portions away from each other in the axial direction, thereby increasing the axial length of the intermediate portion. Both operations, i.e. rolling up and pulling away may be carried out substantially simultaneously or separately (at different times) so expansion of the end portions and decreasing of the length of the intermediate portion may be achieved at different times if necessary.

The linear cut for producing longitudinal edges 326, 327 and the linear cut for producing the longitudinal edges 327, 337 may adopt any relative position to each other so as to obtain a predefined axial expansion of the intermediate portion 340. Put in other words, the relative position of both linear cuts may vary the overall length of the intermediate portion 340 along the helical path and thus the axial expansion/contraction of the anastomosis device may vary. For instance, in implementations of FIGS. 8a, 8b both linear cuts are not aligned with each other but a different relative position may be envisaged.

As mentioned earlier the tubular structure may be made of a shape-memory material, and in this case the operation of activating the tubular structure may include keeping the tubular structure at a temperature lower than the body temperature of about 37° C., for example at room temperature.

When the device is employed in an anastomosis, the structure may inactivate when it is inserted in the body and therefore exposed to the body temperature of about 37° C.

The use of shape-memory material allows some or all the parts of the anastomosis device to self-expand in radial direction and self-contract in axial direction when the device may be subjected to a predetermined temperature, such as body temperature.

Although a number of particular implementations and examples have been disclosed herein, further variants and modifications of the disclosed devices and methods are possible. For example, not all the features disclosed herein are included in all the implementations, and implementations including other combinations of the features described are also possible.

1. An anastomosis device comprising
a tubular structure, said tubular structure comprising
a first tubular end portion,
a second tubular end portion and
an intermediate portion,
the intermediate portion being united at one end to the first tubular end portion and at the other end to the second tubular end portion,
the first and the second tubular end portions each comprising expansion elements to allow the expansion and contraction of each end portion in at least one radial direction thereof, and
the intermediate portion comprising approximation elements configured and attached to the tubular end portions in such a way that they allow the length of the intermediate portion to increase and decrease in the axial direction.

2. A device as claimed in claim 1, the expansion elements being arranged to allow the expansion and contraction of each end portion in a first radial direction in a first dimension while maintaining substantially constant a second dimension of each end portion in a second radial direction perpendicular to the first direction.

3. A device as claimed in claim 1, the expansion elements of the first and second tubular end portions being C-shaped.

4. A device as claimed in claim 1, the expansion elements of the first and second tubular end portions being made of a shape-memory material.

5. A device as claimed in claim 4, the shape-memory material being configured such that the expansion elements can be deformed to compress the end portions in said at least one radial direction at a temperature lower than corporal temperature, and to recover from deformation to expand again the end portions in said at least one radial direction, upon heating.

6. A device as claimed in claim 4, the shape-memory material being one or more of an alloy, or a Nitinol alloy, or a polymer, or a PLLA polymer.

7. A device as claimed in claim 1, the approximation elements of the intermediate portion being made of a shape-memory material.

8. (canceled)

9. (canceled)

10. A device as claimed in claim 1, the surface of each of the first and second tubular end portions comprising at least two plates configured to contact the inner surface of a lumen, the longitudinal edges of two consecutive plates being connected to each other through the expansion elements.

11. A device as claimed in claim 10, the surface of the first and second tubular end portions comprising four plates and four sets of expansion elements.

12. A device as claimed in claim 1, the first and second tubular end portions comprising projections on at least part of their outer surface, the projections being configured for engaging endothelial tissue.

13. A device as claimed in claim 12, the projections on the tubular end portions being spike-shaped and inclined relative to the axial direction of the tubular structure.

14. A device as claimed in claim 13, the projections on each of the first and second tubular end portions being inclined in opposite directions relative to the axial direction of the tubular structure, the opposite directions being configured for promoting insertion of each of the first and second tubular end portions into respective ends of a blood vessel and to oppose withdrawal therefrom.

15. A device as claimed in claim 1, each of the first and the second tubular end portions being configured to be rolled up on themselves.

16. A device as claimed in claim 15, the expansion elements of the first and second tubular end portions comprising a region made of shape-memory material.

17. A device as claimed in claim 15, the approximation elements of the intermediate portion comprising a helical-shaped unit.

18. A method of manufacturing an anastomosis device comprising
a first tubular end portion,
a second tubular end portion and
an intermediate portion,
the intermediate portion being united at one end to the first tubular end portion and at the other end to the second tubular end portion,
the first and the second tubular end portions each comprising expansion elements to allow the expansion and contraction of each end portion in at least one radial direction thereof, and
the intermediate portion comprising approximation elements configured and attached to the tubular end portions in such a way that they allow the length of the intermediate portion to increase and decrease in the axial direction,
the method comprising:
providing a tubular structure;
cutting the tubular structure to form the first and second tubular end portions and the intermediate portion comprising approximation elements configured, and attached to the tubular end portions, to allow the length of the intermediate portion to increase and decrease in the axial direction;
activating the tubular structure to cause a contraction of said first and second tubular end portions in the at least one radial direction and to increase the length of said intermediate portion in an axial direction.

19. A method as claimed in claim 18, the cutting of the tubular structure further comprising:
cutting a linear cut along the surface of the tubular structure in the axial direction extending from a first end to a first point of the tubular structure;
cutting a linear cut along the surface of the tubular structure in the axial direction extending from a second end to a second point of the tubular structure, the second end being opposite to the first end;
cutting a helical-path cut around the axial direction, the helical-path cut being provided between the first and second end of the tubular structure.

20. A method as claimed in claim 18, the activating of the tubular structure further comprising:
rolling the first and second tubular end portions up on themselves;
pulling the first and second tubular end portions away from each other in the axial direction, thereby increasing the axial length of the intermediate portion.

21. A method as claimed in claim 18,
the tubular structure being made of a shape-memory material, and
the activating of the tubular structure further comprising:
keeping the tubular structure at a temperature lower than body temperature.

22. A method for end-to-end vascular anastomosis comprising:
providing an anastomosis device comprising
a first tubular end portion,
a second tubular end portion and
an intermediate portion,
the intermediate portion being united at one end to the first tubular end portion and at the other end to the second tubular end portion,
the first and the second tubular end portions each comprising expansion elements to allow the expansion and contraction of each end portion in at least one radial direction thereof, and
the intermediate portion comprising approximation elements configured and attached to the tubular end portions in such a way that they allow the length of the intermediate portion to increase and decrease in the axial direction;
activating the anastomosis device to cause the first and a second tubular end portions to be compressed in the at least one radial direction and the length of the intermediate portion to increase in the axial direction;
inserting the first tubular end portion in a first blood vessel and the second tubular end portion in a second blood vessel respectively the first and second blood vessels being disposed to be anastomosed to each other; and
inactivating the device to allow the first and a second tubular end portions to expand in said at least one radial direction and allowing the length of the intermediate portion to decrease such that the end portions are pulled towards each other.
